(19) United States
(12) Patent Application Publication
(10) Pub. No.: US 2006/0182823 A1
(43) Pub. Date: Aug. 17, 2006

(54) A process for preparing lotus leaf extracts and method of use

(75) Inventors: Lin Yang, Chengdu (CN); Jie Fang, Chengdu (CN); Xiaoqiang Zheng, Chengdu (CN)

Correspondence Address: SAL ABRAHAM, 1470 MOHAWK LANE, EASTON, PA, 18040, US

(73) Assignee: CHENGDU WAGOTT NATURAL PRODUCTS CO., LTD., Chengdu (CN)

(21) Appl. No.: 10/906,294

(22) Filed: Feb. 13, 2005

(51) Int. Cl. A61K 36/48

(52) U.S. Cl. 424/757

(57) ABSTRACT

This invention provides a process for extracting the flavonoid and alkaloid components from lotus leaf and provides a method for the reduction of body weight, total cholesterol (TC), low density lipoprotein (LDL) cholesterol, and triglycerides (TG). It also provides a method for increasing high density lipoprotein (HDL) cholesterol and the prevention of coronary heart disease (CHD). There are 10% to 80% flavones and 10% to 80% alkaloids in the lotus leaf extract powder of this invention. This process produces an improved standardized raw material that may be utilized as a single dietary supplement, food additive, or medicine and it may be added to other plant extracts, nutraceuticals, or pharmaceuticals.

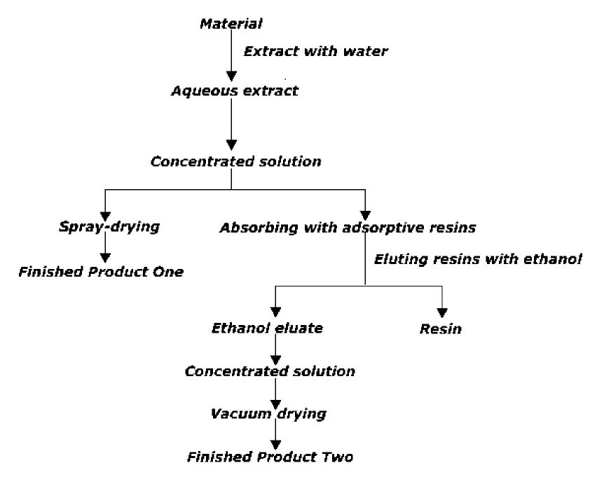

### BACKGROUND OF INVENTION

Now more than ever with the improvement in the standard of living and the increased consumption of high calorie foods, there are more and more people who suffer from obesity and abnormal blood lipids, which are both recognized as major risk factors for the development of coronary heart disease (CHD).

Lotus leaf is the leaf of nelumbo nuciferea gaertn, it is a traditional Chinese herb with the function of reducing weight and fat content. Lotus leaf has many dosage form applications including oral, sublingual, transdermal, nasal, and parenteral. In lotus leaf, the flavone and alkaloid have biologic activity and physiological function Du Lijun, et al. Chinese Traditional and Herbal Drugs, 2001, Vol. 31, 7:526-528 studied the effect of the aqueous non-standardized extracts of lotus leaf on serum total cholesterol (TC) and triglyceride (TG) with acute hyperlipemic mouse model. This research suggests aqueous extracts of lotus leaf can regulate blood lipid levels. Xu Laying, et al. Journal of Hubei Chinese medicine (in Chinese), 1996, 4: 42-43 has demonstrated that non-standardized lotus leaf extracts can lower blood lipid levels by mice feeding test.

CN patent 1435361 to Hu Shilin et al. discloses a method for a natural health-care beverage-cold weight-reducing lotus throne juice. It is made of natural plant lotus leaf, bamboo leaf, peppermint and lotus plumule through a certain preparation process. Said invented beverage has the functions of quenching thirst, removing heat, relieving restlessness, clearing away the heart-fire and reducing weight. However, while this may be an improvement in functional beverages it does not contain a standardized extract of lotus leaf and therefore does not represent an improvement Lotus Leaf applications.

U.S. Pat. No. 5,958,417 to Hsu; Chau-shin discloses herbal combinations which reduce serum cholesterol and triglyceride levels comprise (1) herbs having substantial recognized activity in enhancing circulatory function (ECF herbs) and (2) herbs having substantial recognized effects in promoting bowel motility (PBM herbs). Preferred combinations have a combined total of at least 16% (dry weight basis) of at least two ECS herbs in combination with a combined total of at least 16% (dry weight basis) of at least two PBM herbs. Particularly preferred combinations are selected from the following herbs: Crataegus, Ho Shou Wu, Chrysanthemum, Lotus Leaf, Alisma and Hu-Zhang, Cassia Seed, and Rhubarb. However, while this may be an improvement in herbal combinations it does not contain a standardized extract of lotus leaf and therefore does not represent an improvement Lotus Leaf applications.

U.S. Patent application 20040044079 A1 discloses methods of promoting weight control in a companion animal comprising orally administering one or more non-glyceryl derivatives of C.sub.17 or greater fatty acids. Also disclosed are methods for promoting weight control in a human comprising orally administering non-glyceryl derivatives of C.sub.17 or greater fatty acids, wherein the fatty acid derivatives do not cause the human to reduce food consumption. Further disclosed are methods for promoting weight control in a human or companion animal comprising orally administering lotus leaf extract. Further disclosed are dietary compositions for promoting weight control in a companion animal, wherein such compositions comprise one or more of the non-glyceryl derivatives of C.sub.17 or greater fatty acids and the lotus leaf extract. However, while this may be an improvement in weight control combinations it does not contain a standardized extract of lotus leaf and therefore does not represent an improvement Lotus Leaf applications.

The most traditional application of lotus leaf is in the form of tea, such as Sanhua Jianfei tea, Wulong Jianfei tea, Jiangzhi Jianfei tea, etc. To the best of our knowledge there is no patent, which utilizes this novel process of standardization for the lotus leaf extracts of this invention alone in health care food, medicine, or any application to prevent or treat hyperlipidemia and obesity. The lotus leaf extracts of this invention are superior to all previous extracts due to the high percentage of standardized active flavone and alkaloid components.

### SUMMARY OF INVENTION

This invention provides a process for extracting flavonoid and alkaloid components from lotus leaf. These components maybe utilized as a dietary supplement, food additive or material to make health care foods or medicines. This invention can also be utilized to prevent and treat hyperlipidemia, hypertriglyceridemia, and CHD. This novel and unobvious invention utilizes standardized active ingredients of the lotus leaf for superior function.

### DETAILED DESCRIPTION

The invention provides a process for extracting components, which mainly consist of flavonoids and alkaloids from lotus leaf with a novel technique, easy manipulation and inexpensive production cost. The extracts can be used as a main component or food additive in health care food or medicine.

The extracts of the invention are obtained by using the technical scheme as follows:

The process mainly includes extracting with water, filtering the extract solution, concentrating the solution, moving the resin into column, eluting with ethanol, concentrating, and drying this eluate.

Laboratory analysis of the effective part of the lotus leaf extracts, include one or several flavonoid components such as Quercetin, Isoquercitrin, Nelumboside. The alkaloid components include Nuciferine, Roemerine, Nornuciferine, Anonaine, Pronuciferine, N-N ornuciferine, Liriodenine, Dehydroroemerine, Dehydronuciferine, and Dehydroanonaine.

These flavonoid and alkaloid components extracted from lotus leaf can be used to make health care foods or medicines, which possess blood lipid regulating and treating effects.

These extracts can be used alone or mixed with other Chinese herbs to reduce weight and fat content in mammals such as animals and humans. They can be used to prevent and treat CHD, which can be induced by abnormal blood lipids.

Other Chinese herbs' extracts may consist of one or several components of all those herbs which have the function of reducing weight and fat, such as Mulberry Leaf extract, Hawthorn Fluid extract, Szechuan Lovage Rhizome extract, Angelica extract, Sanchi extract, etc.

A clinical animal study was undertaken by the inventors in order demonstrate the physiological functions of the standardized Lotus Leaf extract of this invention. Choosing 80 neonatal km mice, and given sodium glutamate for continues hypodermic injection for 5 days, after weaning, the 80 km mice were raised by basic food and combined with a normal group of 10 same week age mice and the quantity of food is unlimited. After 4 weeks, the weight of model group was significantly heavier than the normal group of the same week age mice. They then selected 50 model mice whose weight was in accordance with the standard. Then based on weight, the model group was randomly divided into 5 groups with 10 cases and both male and female in each group with one control group and four treatment groups. The four treatment groups include a high dosage group of extract (A), a low dosage group of extract (A), a high dosage group of extract (B), and a low dosage group of extract (B). There is 20% flavonoid in extract (A) and 40% in extract (B). The dose of the high dosage group is 20 times than the clinical dosage for humans, and the dosage of the low dosage group is 5 times than the clinical dosage for humans. The mice of the four treatment groups were executed after given medicine for 30 days, then the correlative targets were detected.

Before treatment, the weight of the model group was significantly heavier than that of the normal group (P<0.01). After treatment, the Lee's exponent and the Fat exponent of the control group were significantly higher than those of the normal group (P<0.01), and the weight of the control group was far heavier than that of the normal group (P<0.05). Comparing with the control group, the Fat exponent of the low dosage group of extract (A) was significantly decreased (P<0.01), and the Lee's exponent of the low dosage group of extract (A) was greatly reduced (P<0.05). The weight of the high dosage group of extract (A) was far lighter than that of the control group (P<0.05), the Lee's exponent of the high dosage group of extract (A) was greatly reduced (P<0.01), and the Fat exponent of the low dosage group of extract (B) was significantly decreased (P<0.05). The Lee's exponent of the low dosage group of extract (B) was greatly reduced (P<0.05), and the Fat exponent of the low dosage group of extract (B) was significantly decreased (P<0.01). The Lee's exponent and the Fat exponent of the high dosage group of extract (B) were both greatly reduced (P<0.01).

Comparing the blood biochemistries of the control group in the low dosage group of extract (A), TC and TG were significantly lower (P<0.001), high density lipoprotein (HDL) was greatly increased (P<0.001), low density lipoprotein (LDL) was significantly decreased (P<0.05); In the high dosage group of extract (A), TC was significantly decreased (P<0.001), TG and LDL were greatly decreased (P<0.05); In the low dosage group of extract (B), TC and TG were significantly decreased (P<0.05), HDL was greatly increased (P<0.001), LDL was greatly decreased (P<0.001).

The following data illustrates the effects on the bulk of the lipocyte: The low dosage group of extract (A), the perimeter and the area of lipocyte was greatly reduced (P<0.0010), the quantity of lipocyte in unit visual field was greatly increased (P<0.001); In the high dosage group of extract (A), the perimeter and the area of lipocyte was greatly reduced (P<0.001), the quantity of lipocyte in unit visual field was greatly increased (P<0.001); In the high dosage group of extract (B), the perimeter of lipocyte was greatly reduced (P<0.001), the area of lipocyte was greatly reduced (P<0.005), and the quantity of lipocyte in unit visual field was greatly increased (P<0.001).

The following examples should not be considered as limitations of the present invention.

### EXAMPLE 1

Cutting the dry fresh lotus leaves into small segments, extracting 100 grams crushed leaves with 2000 milliliters hot water at a temperature of 50 degrees Celsius., then extract 2 to 4 times and each time lasts 20 to 40 minutes. Then combine this extract solution, filter and concentrate the extract solution with a vacuum under heat at a temperature of 50 degrees Celsius. Spray-dry the concentrated solution directly to the 60 grams of finished product providing about 20% lotus flavones.

### EXAMPLE 2

Cutting the dry fresh lotus leaves into small segments, extract 100 grams lotus leaves with 2000 milliliters hot water at a temperature of 50 degrees Celsius. for 30 minutes, twice. Combine the solutions and absorb active compounds with the resin (D101) and then move the resin into column. Elute with water till the color of the percolate changes into straw yellow. Then elute with 70% ethanol till the color turns into buff. Finally concentrate with vacuum under heat at a temperature of 50 degrees Celsius and dry the solution to 2.5 grams of product at about 40% lotus flavones.

The foregoing descriptions of the invention are for illustration only. Modifications not included in the description, which are obvious to those skilled in the art, are intended to be included in the scope of the claims.

What is claimed is:

1. A process for preparing Lotus leaf extract, which comprises:
a) Drying and crushing fresh lotus leaves. The crushed leaves are extracted with water or solvent, and then filtered to keep the extract solution.
b). Concentrate the solution from step a) under vacuum heat and absorb active components with resin.
c). Move these resins into column and add solvent.
d). Concentrate the solution from step c) under vacuum heat and dry to powder.

2. The process according to claim 1a, wherein the water temperature is between 50 to 100 degrees Celsius.

3. The process according to claim 1a and 1c, wherein the solvent is selected from the group consisting of water, 10% to 90% ethanol, methanol, butanol, and propanol.

4. The process according to claim 1b and 1d, wherein the vacuum heat temperature is between 40 to 100 degrees Celsius.

5. The process according to claim 1b, wherein the active components are flavones and alkaloids ranging from 5% to 80%.

6. The process according to claim 1b and 1d, wherein the said extract is concentrated under reduced pressure.

7. The process according to claim 1b, wherein the resin is a macro porous adsorptive resin selected form the group consisting of macro-reticular resin made of polyvinyl or polypropylene.

8. A method of administrating to a human or animal an effective amount of Lotus Leaf extract for the reduction of body weight.

9. A method of administrating to a human or animal an effective amount of Lotus Leaf extract for the reduction of serum total cholesterol and for treating and preventing coronary heart disease.

10. A method of administrating to a human or animal an effective amount of Lotus Leaf extract for the reduction of serum low density lipoprotein cholesterol.

11. A method of administrating to a human or animal an effective amount of Lotus Leaf extract for the reduction of serum triglycerides.

12. A method of administrating to a human or animal an effective amount of Lotus Leaf extract for increasing high density lipoprotein cholesterol.
